SEQUENCE LISTING (16 sequences)

SEQ ID NO: 1 - protein, 305 aa, synthetic construct
  REGION 1..305: note = Synthetic Sequence
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 2 - protein, 275 aa, synthetic construct
  REGION 1..275: note = Synthetic Sequence
MSRELAPLLLLLLSIHSALAMRICSFNVRSFGESKQEDKNAMDVIVKVIKRCDIILVMEIKDSNNRICPILMEKLNREKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 3 - protein, 23 aa, synthetic construct
  REGION 1..23: note = Synthetic Sequence
SSRAFTNSKKSVTLRKKTKSKRS

SEQ ID NO: 4 - protein, 585 aa, synthetic construct [sequence omitted]

SEQ ID NO: 5 - protein, 81 aa, synthetic construct
  REGION 1..81: note = Synthetic Sequence
MRFPSIFTAVLFAASSALAAPVNTTTEDETAQIPAEAVIGYSDLEGDFDVAVLPFSNSTNNGLLFINTTIASIAAKEEGVS

SEQ ID NO: 6 - protein, 24 aa, synthetic construct
  REGION 1..24: note = Synthetic Sequence
MKWVTFISLLFLFSSAYSRGVFRR

SEQ ID NO: 7 - protein, 20 aa, synthetic construct
  REGION 1..20: note = Synthetic Sequence
MSRELAPLLLLLLSIHSALA

SEQ ID NO: 8 - protein, 5 aa, synthetic construct
  REGION 1..5: note = Synthetic Sequence
GGGGS

SEQ ID NO: 9 - protein, 15 aa, synthetic construct
  REGION 1..15: note = Synthetic Sequence
GGGGSGGGGSGGGGS

SEQ ID NO: 10 - protein, 14 aa, synthetic construct
  REGION 1..14: note = Synthetic Sequence
APAPAPAPAPAPAP

SEQ ID NO: 11 - protein, 12 aa, synthetic construct
  REGION 1..12: note = Synthetic Sequence
AEAAAKEAAAKA

SEQ ID NO: 12 - protein, 7 aa, synthetic construct
  REGION 1..7: note = Synthetic Sequence
SGGSGSS

SEQ ID NO: 13 - protein, 16 aa, synthetic construct
  REGION 1..16: note = Synthetic Sequence
SGGSGGSGGSGGSGSS

SEQ ID NO: 14 - protein, 31 aa, synthetic construct
  REGION 1..31: note = Synthetic Sequence
SGGSGGSGGSGGSGGSGGSGGSGGSGGSGSS

SEQ ID NO: 15 - protein, 29 aa, synthetic construct
  REGION 1..29: note = Synthetic Sequence
GGSGGSGGSGGSGGSGGSGGSGGSGGSGS

SEQ ID NO: 16 - protein, 7 aa, synthetic construct
  REGION 1..7: note = Synthetic Sequence
DVFSREP

(19) United States
(12) Patent Application Publication
(10) Pub. No.: US 2025/0179451 A1
(43) Pub. Date: Jun. 5, 2025

(54) DNASE ENZYMES ENGINEERED FOR IMPROVED STABILITY

(71) Applicant: NEUTROLIS, INC., Cambridge, MA (US)

(72) Inventor: Toby FOX, Cambridge, MA (US)

(21) Appl. No.: 18/840,667

(22) PCT Filed: Feb. 23, 2023
(86) PCT No.: PCT/US2023/013680

§ 371 Date: Aug. 22, 2024

Related U.S. Application Data
(60) Provisional application No. 63/326,499, filed on Apr. 1, 2022.
(60) Provisional application No. 63/312,891, filed on Feb. 23, 2022.

(51) Int. Cl. C12N 9/22; A61K 38/00; A61K 47/60

(52) U.S. Cl. CPC C12N 9/22; A61K 47/60; A61K 38/00; C07K 2319/30; C07K 2319/31; C12Y 301/21001

(57) ABSTRACT

The present disclosure provides D1L3 enzymes with reduces susceptibility to proteolysis and/or which provide improved in vivo stability or half-life. In accordance with aspects of the disclosure, the D1L3 enzymes described herein are more physiologically stable and thus are suitable for therapy with reduced doses and/or less frequent dosing. In embodiments, the D1L3 enzymes have benefits for systemic therapy, which include longer exposure and extended duration of pharmacodynamic action.

### PRIORITY

The present application claims the benefit of, and priority to, U.S. provisional application No. 63/312,891 filed Feb. 23, 2022, and U.S. provisional application No. 63/326,499 filed Apr. 1, 2022, the contents of which are hereby incorporated by reference in their entireties.

### FIELD OF THE INVENTION

The present disclosure provides, in part, DNase enzymes engineered for improved stability and/or circulatory half-life for use in therapy.

### DESCRIPTION OF THE TEXT FILE SUBMITTED ELECTRONICALLY

This application contains a sequence listing. It has been submitted electronically via EFS-Web as an ASCII text file entitled “NTR-013PR_2.txt.” The sequence listing is 14,395 bytes in size, and was created on Mar. 23, 2022. The sequence listing is hereby incorporated by reference in its entirety.

### BACKGROUND

Enzyme replacement therapy with DNase enzymes is a promising approach for treatment of chronic diseases including but not limited to systemic lupus erythematosus (SLE). However, DNase enzymes such as DNase1-like 3 (D1L3) do not exhibit high circulatory half-life in-part because they are susceptible to proteolytic degradation. Accordingly, engineered enzymes, including D1L3 enzymes, with high protease resistance and extended in vivo half-life are desired.

### SUMMARY OF THIS DISCLOSURE

The present disclosure is based, in part, on identification of D1L3 amino acid sequences that are susceptible to proteolysis and/or which provide opportunities to improve in vivo stability or half-life. In accordance with aspects of the disclosure, the D1L3 enzymes described herein are more physiologically stable and thus are suitable for therapy with reduced doses and/or less frequent dosing. In embodiments, the D1L3 enzymes have benefits for systemic therapy, which include longer exposure and extended duration of pharmacodynamic action.

In one aspect, the present disclosure provides a D1L3 enzyme comprising a modification that reduces proteolysis after the amino acid corresponding to F130 of SEQ ID NO: 1 or the amino acid corresponding to F100 of SEQ ID NO: 2. In embodiments, the D1L3 enzyme comprises an amino acid sequence that has at least 80% sequence identity to SEQ ID NO: 1 or SEQ ID NO: 2.

In embodiments, the D1L3 comprises one or more amino acid substitutions, insertions, and/or deletions within the sequence D128 to P134, numbered with respect to SEQ ID NO: 1. In embodiments, the D1L3 comprises one or more amino acid substitutions, insertions, and/or deletions within the sequence D98 to P104 of SEQ ID NO: 2. In embodiments, the substitutions, insertions, and/or deletions remove a protease site from D1L3. In embodiments, the one or more modifications disrupt a protease consensus sequence in D1L3. In embodiments, the substitutions, insertions, and/or deletions block access of a protease site to a cleavage site in D1L3. In embodiments, the D1L3 enzyme comprises one, two, three, four, five or more amino acid modifications independently selected from substitutions, deletions, and insertions in the sequence corresponding to D128 to P134 of the SEQ ID NO: 1 (e.g., in the sequence corresponding to amino acids D98 to P104 of SEQ ID NO: 2). In embodiments, this region of the engineered D1L3 enzyme is more resistant to chymotrypsin-like proteases and/or serine proteases.

In embodiments, the amino acid F130 corresponding to SEQ ID NO: 1 is substituted for a different amino acid. In embodiments, the modification comprises an amino acid substitution at S131 with respect to SEQ ID NO: 1. In such embodiments, the amino acid corresponding to S131 of SEQ ID NO: 1 is substituted with any amino acid that blocks the protease cleavage at the peptide bond that precedes the amino acid.

In some embodiments, the modification is the conjugation of a bulky group to the D1L3 enzyme that blocks cleavage of the peptide bond joining amino acids corresponding to F130 and S131 of SEQ ID NO: 1 by a protease. In embodiments, the bulky group is conjugated at the position corresponding to F130 and/or S131 of SEQ ID NO: 1. In embodiments, the bulky group is conjugated at the position corresponding to 1, or 2, or 3, or 4, or 5 amino acids away from F130 and/or S131 of SEQ ID NO: 1. Exemplary suitable bulky groups are independently selected from glycosyl groups, acyl groups, and polymers such as polyethylene glycol (PEG).

In another aspect, the present disclosure provides a D1L3 enzyme comprising a modification that reduces proteolysis after the amino acid corresponding to R95 of SEQ ID NO: 1. In embodiments, the D1L3 enzyme comprises an amino acid sequence that has at least 80% sequence identity to SEQ ID NO: 1. In embodiments, the D1L3 comprises one or more amino acid substitutions, insertions, and/or deletions within the sequence R92 to E100 of SEQ ID NO: 1. In embodiments, the substitutions, insertions, and/or deletions remove a protease site from D1L3. In embodiments, the one or more modifications disrupt a protease consensus sequence in D1L3. In embodiments, the substitutions, insertions, and/or deletions block access of a protease site to a cleavage site in D1L3. In embodiments, the D1L3 enzyme comprises one, two, three, four, five or more amino acid modifications independently selected from substitutions, deletions, and insertions in the sequence corresponding to R92 to E100 of the SEQ ID NO: 1. In embodiments, the D1L3 enzyme of this disclosure is more stable (resistant to proteolysis) in expression systems, including those described herein (e.g., Pichia pastoris). In embodiments, the D1L3 enzyme is more stable (resistant to proteolysis) in serum.

In embodiments, the amino acid R95 corresponding to SEQ ID NO: 1 is substituted for a different amino acid. In embodiments, the modification comprises an amino acid substitution at N96 with respect to SEQ ID NO: 1. In some embodiments, the modification is the conjugation of a bulky group to the D1L3 enzyme that blocks cleavage of the peptide bond joining amino acids corresponding to R95 and N96 of SEQ ID NO: 1 by a protease. In embodiments, the bulky group is conjugated at the position corresponding to R95 and/or N96 of SEQ ID NO: 1. In embodiments, the bulky group is conjugated at the position corresponding to 1, or 2, or 3, or 4, or 5 amino acids away from R95 and/or N96 of SEQ ID NO: 1.

In some embodiments, one or more non-cysteine (Cys of C) residues are mutated to a Cys and PEGylated (e.g., by site-specific PEGylation). Such additional sites for PEGylation can be additional proteolytically susceptible sites in wild type D1L3. In these or other embodiments, the amino acid C68 relative to SEQ ID NO: 1, which is believed to be unpaired and accessible in wild-type D1L3, is PEGylated or otherwise modified. For example, the amino acid corresponding to C68 of SEQ ID NO: 1 can be substituted with another amino acid (e.g., Ala, Ser, or Gly, or any amino acid other than Cys), to thereby remove this unpaired Cysteine and/or prevent undesired PEGylation at this position. In still other embodiments, the amino acid C68 relative to SEQ ID NO: 1 forms a disulfide bond with a Cys substituted at a different position. In some embodiments, a Cys is substituted at a position selected from I60, Y87, I89, A103, and L105 relative to SEQ ID NO: 1, and forms a disulfide bond with the side chain of C68. In some embodiments, the modifications to the D1L3 enzyme avoid disulfide scrambling and/or protein misfolding.

In some embodiments, a PEG moiety will provide a half-life extension property and/or improved stability, by providing resistance to proteolysis, reduced disulfide scrambling and/or protein misfolding, and/or a larger hydrodynamic radius to reduce clearance from circulation.

In embodiments, the D1L3 enzyme is a fusion protein with a half-life extending polypeptide (e.g., a carrier protein). In embodiments, the D1L3 enzyme is fused to a carrier protein optionally by means of an amino acid linker. The carrier protein in some embodiments is selected from albumin, transferrin, an Fc domain, XTEN, or elastin-like protein, or a variant thereof.

In embodiments, the D1L3 enzyme is fused to an albumin amino acid sequence or domain, i.e., a human albumin or a fragment or variant thereof. Albumin can be joined to the D1L3, optionally with an interposed linker, at the N-terminus and/or the C-terminus of the D1L3 enzyme. An exemplary albumin amino acid sequence is provided by SEQ ID NO: 4. In embodiments, the D1L3 enzyme comprises an albumin sequence fused to the N-terminus and/or C-terminus of the mature D1L3 enzyme with an interposed amino acid linker. Linker constructs are described in detail herein.

In embodiments, the D1L3 enzyme is fused to an Fc domain. In embodiments, the human Fc domain is selected from IgG1, IgG2, IgG3, and IgG4. In embodiments, the human Fc domain is a human IgG Fc domain. In embodiments, the Fc domain has at least two heavy chain constant region domains (e.g., CH2 and CH3) and a hinge region. The Fc domain can be joined to the D1L3, optionally with an interposed linker, at the N-terminus and/or the C-terminus of the D1L3 enzyme. In embodiments, the D1L3 enzyme comprises an Fc domain sequence fused to the N-terminus of the mature D1L3 enzyme with an interposed amino acid linker. In embodiments, the D1L3 enzyme comprises an Fc domain sequence fused to the C-terminus of the D1L3 enzyme, optionally through a linker (e.g., a flexible linker). In embodiments, the D1L3 enzyme (with Fc fused at the C-terminus) has a mutation in one or more amino acids of the BD that reduces or eliminates proteolytic cleavage (without limitation, e.g., substitutions of paired basic amino acids). In embodiments, the D1L3 enzyme (with Fc fused at the C-terminus) has a deletion of the BD to remove the paired basic amino acids in the BD that are otherwise substrates for proteolytic cleavage.

In one aspect, the present disclosure provides an isolated polynucleotide encoding the D1L3 enzyme of any of embodiments disclosed herein. In embodiments, the polynucleotide is used for expression of the D1L3 enzyme in host cells suitable for expressing the D1L3 enzyme, and optionally adding glycosyl moieties as described herein for certain embodiments. In other embodiments, the recombinant D1L3 enzyme is expressed and recovered from a recombinant expression system (as described herein), and PEGylated ex vivo using the desired conjugation chemistry. In one aspect, the present disclosure provides a vector comprising the polynucleotide of any of embodiments disclosed herein. In other embodiments, the polynucleotide is used for therapy, such as gene therapy or mRNA therapy.

In one aspect, the present disclosure provides a pharmaceutical composition comprising the D1L3 enzyme of any of embodiments disclosed herein, and a pharmaceutically acceptable carrier. In embodiments, the pharmaceutical composition is formulated for parenteral delivery, such as for intravenous or subcutaneous administration.

In other aspects, the present disclosure provides a method for treating a subject in need of extracellular chromatin degradation, extracellular trap (ET) degradation and/or neutrophil extracellular trap (NET) degradation. The method comprises administering a therapeutically effective amount of the D1L3 enzyme or composition described herein. In embodiments, the present invention provides a method for treating, preventing, or managing diseases or conditions characterized by the presence or accumulation of NETs. A number of stimuli, which sometimes contribute to inflammation and/or pathogenesis, induce NETs. The diseases or conditions characterized by the presence or accumulation of NETs include, but are not limited to, diseases associated with chronic neutrophilia, neutrophil aggregation and/or leukostasis, thrombosis and vascular occlusion, ischemia-reperfusion injury, surgical and traumatic tissue injury, an acute or chronic inflammatory reaction or disease, an autoimmune disease, cardiovascular disease, metabolic disease, systemic inflammation, inflammatory diseases of the respiratory tract, renal inflammatory diseases, inflammatory diseases related to transplanted tissue or hematopoietic stem cell transplantation (e.g. graft-versus-host disease), inflammation caused by viral infections (e.g. COVID-19), and cancer (including leukemia). In embodiments, the present invention provides a method for treating complete or partial vascular or ductal occlusions involving extracellular chromatin, and including NETs. In embodiments, the disease or condition is an autoimmune or immunological condition, such as but not limited to systemic lupus erythematosus (SLE).

In embodiments, the present invention pertains to the treatment of diseases or conditions characterized by deficiency of D1L3, or a deficiency of D1. In some cases, the subject has a mutation (e.g., a loss of function mutation) in a Dnase113 gene or a Dnase1 gene. Such subjects can manifest with an autoimmune disease, such as: systemic lupus erythematosus (SLE), lupus nephritis, scleroderma or systemic sclerosis, rheumatoid arthritis, inflammatory bowel disease, Crohn's disease, ulcerative colitis, and urticarial vasculitis.

Other aspects and embodiments of the invention will be apparent from the following detailed description.

### BRIEF DESCRIPTION OF THE DRAWINGS

FIGS. 1A-1B show SDS-PAGE analysis of purified DNASE1L3 produced in Pichia pastoris. Full Length D1L3 and two fragments (labeled as “Fragment 1” and “Fragment 2”) are identified (FIG. 1A). FIG. 1B shows the amino acid sequences of C-terminal sequence of Fragment 1 and N-terminal sequence of Fragment 2 and a reconstruction of a cleavage site. This site is identified as a cleavage site for chymotrypsin-like proteases.

### DETAILED DESCRIPTION

The present disclosure is based, in part, on identification of D1L3 amino acid sequences that are susceptible to proteolysis and/or which provide opportunities to improve in vivo stability or half-life. In accordance with aspects of the disclosure, the D1L3 enzymes described herein are more physiologically stable (e.g., more stable in serum) and thus are suitable for therapy with reduced doses and/or less frequent dosing. In embodiments, the D1L3 enzymes have benefits for systemic therapy, which include longer exposure (e.g., slower elimination, longer circulatory half-life, and reduced susceptibility to proteolysis), and extended duration of pharmacodynamic action.

In the description that follows, certain conventions will be followed regarding the usage of terminology. As used herein and in the claims, the singular forms “a,” “an,” and “the” include the singular and the plural reference unless the context clearly indicates otherwise.

As used herein, unless stated to the contrary, the term “D1L3” when referring to the wild-type sequence, includes either D1L3 Isoform 1 (SEQ ID NO: 1) or D1L3 Isoform 2 (SEQ ID NO: 2).

When referring to sequence identity with wild-type DNase enzymes, and unless stated otherwise, amino acid sequences refer to mature enzymes lacking the signal peptide. Further, unless stated otherwise, amino acid positions are numbered with respect to the full-translated DNase sequence, including signal peptide, for clarity. Accordingly, for example, reference to sequence identity to the enzyme of SEQ ID NO: 1 (human D1L3, Isoform 1) refers to a percent identity with the mature enzyme having M21 at the N-terminus.

As used herein with reference to a drug, “half-life” refers to the elimination half-life of the concentration of the drug in an animal, as measured in a matrix of interest, e.g., serum or plasma. The skilled person will understand that not all drugs exhibit first-order kinetics or do so during all phases of elimination. In such cases, the skilled person will understand that the terms “half-life extension” or “extended half-life” are expressions that refer to a slower rate of elimination.

As used herein, “neutrophil extracellular trap” and the acronym “NET” refer to a network of extracellular fibers comprising nuclear contents, e.g., DNA bound to histone proteins that are released from an immune cell, typically a neutrophil, in a programmed fashion.

Unless otherwise specified, a “nucleotide sequence” or “nucleic acid” encoding an amino acid sequence includes all degenerate versions that encode the same amino acid sequence. The phrase nucleotide sequence that encodes a protein or an RNA may also include introns to the extent that the nucleotide sequence encoding the protein may in some versions contain one or more introns.

The terms “about” and “approximately” include an amount that is +10% of an associated numerical value.

In one aspect, the present disclosure provides a D1L3 enzyme comprising a modification that reduces proteolysis after the amino acid corresponding to F130 of SEQ ID NO: 1 or the amino acid corresponding to F100 of SEQ ID NO: 2. In embodiments, the D1L3 enzyme comprises an amino acid sequence that has at least 80%, or at least about 85%, or at least about 90%, or at least about 95%, or at least about 96%, or at least about 97%, or at least about 98%, or at least about 99%, sequence identity to SEQ ID NO: 1 or SEQ ID NO: 2.

In embodiments, the D1L3 comprises one or more amino acid substitutions, insertions, and/or deletions within the sequence D128 to P134, numbered with respect to SEQ ID NO: 1. In embodiments, the D1L3 comprises one or more amino acid substitutions, insertions, and/or deletions within the sequence D98 to P104 of SEQ ID NO: 2. In embodiments, the substitutions, insertions, and/or deletions remove a protease site from D1L3. In embodiments, the one or more modifications disrupt a protease consensus sequence in D1L3. In embodiments, the substitutions, insertions, and/or deletions block access of a protease site to a cleavage site in D1L3. In embodiments, the D1L3 enzyme comprises one, two, three, four, five or more amino acid modifications independently selected from substitutions, deletions, and insertions in the sequence corresponding to D128 to P134 of the SEQ ID NO: 1 (e.g., in the sequence corresponding to amino acids D98 to P104 of SEQ ID NO: 2). In embodiments, this region of the engineered D1L3 enzyme is more resistant to chymotrypsin-like proteases and/or serine proteases. In embodiments, the D1L3 enzyme of this disclosure is more stable (resistant to proteolysis) in expression systems, including those described herein (e.g., Pichia pastoris). In embodiments, the D1L3 enzyme is more stable (resistant to proteolysis) in serum.

In embodiments, the amino acid F130 corresponding to SEQ ID NO: 1 is substituted for a different amino acid. In some embodiments, the amino acid corresponding to F130 of SEQ ID NO: 1 is substituted with any amino acid other than an aromatic amino acid. In some embodiments, the amino acid is not leucine. In various embodiments, the amino acid corresponding to F130 of SEQ ID NO: 1 is substituted with a polar, charged, or a non-aliphatic amino acid. For example, in some embodiments, the amino acid corresponding to F130 of SEQ ID NO: 1 is substituted with an amino acid selected from serine (Ser or S), threonine (Thr or T), cysteine (Cys or C), glutamine (Glu or Q), and asparagine (Asn or N). In some embodiments, the amino acid corresponding to F130 of SEQ ID NO: 1 is substituted with an amino acid selected from lysine (Lys or K), arginine (Arg or R), aspartic acid (Asp or D) and glutamic acid (Glu or E). In some embodiments, the amino acid corresponding to F130 of SEQ ID NO: 1 is substituted with an amino acid selected from glycine (Gly or G), alanine (Ala or A), valine (Val or V), isoleucine (Ile or I), proline (Pro or P) and methionine (Met or M). In some embodiments, the amino acid corresponding to F130 of SEQ ID NO: 1 is substituted with Ala, Gly, Thr, or Pro.

In embodiments, the modification comprises an amino acid substitution at S131 with respect to SEQ ID NO: 1. In such embodiments, the amino acid corresponding to S131 of SEQ ID NO: 1 is substituted with any amino acid that blocks the protease cleavage at the peptide bond that precedes the amino acid. In some embodiments, the modification comprises a substitution of the amino acid corresponding to S131 of SEQ ID NO: 1 with proline (Pro or P). Other suitable amino acids include Ala, Gly, Leu, Val, and Ile.

In various embodiments, the protease cleavage site described herein is not altered by introduction of a Cysteine, which can allow for undesired disulfide bond formation (intramolecular or intermolecular).

In some embodiments, the modification is the conjugation of a bulky group to the D1L3 enzyme that blocks cleavage of the peptide bond joining amino acids corresponding to F130 and S131 of SEQ ID NO: 1 by a protease. In embodiments, the bulky group is conjugated at the position corresponding to F130 and/or S131 of SEQ ID NO: 1. In embodiments, the bulky group is conjugated at the position corresponding to 1, or 2, or 3, or 4, or 5 amino acids away from F130 and/or S131 of SEQ ID NO: 1. In embodiments, the bulky group is conjugated at the position corresponding to less than about 15, or less than about 12, or less than about 10, or less than about 8, or less than about 6, or less than about 5 amino acids away from F130 and/or S131 of SEQ ID NO: 1. In embodiments, the bulky group is conjugated at or between the positions corresponding to R115 and V146 of SEQ ID NO: 1. In embodiments, the bulky group is conjugated at or between the positions corresponding to H120 to S141 of SEQ ID NO: 1. In embodiments, the bulky group is conjugated at or between the positions corresponding to G125 to V137 of SEQ ID NO: 1. In embodiments, the bulky group is conjugated at or between the positions corresponding to D128 to P134 of SEQ ID NO: 1. In some embodiments, the bulky group is not conjugated at the position corresponding to S131 of SEQ ID NO: 1.

In embodiments, the modification is the conjugation of at least one, or at least two, or at least three, or at least four, or at least five a bulky groups to the D1L3 enzyme that block cleavage of the peptide bond joining amino acids corresponding to F130 and S131 of SEQ ID NO: 1 by a protease. In embodiments, the bulky groups are conjugated at positions selected from those corresponding to F130, S131, and at one or more positions within 10 amino acids away from F130 and/or S131 of SEQ ID NO: 1 (in either direction). In embodiments, the at least one, or at least two, or at least three, or at least four, or at least five bulky groups are conjugated at or between the position corresponding to R115 and V146 of SEQ ID NO: 1. In embodiments, the at least one, or at least two, or at least three, or at least four, or at least five bulky groups are conjugated at or between the positions corresponding to H120 to S141 of SEQ ID NO: 1. In embodiments, the at least one, or at least two, or at least three, or at least four, or at least five bulky groups are conjugated at or between the positions corresponding to G125 to V137 of SEQ ID NO: 1. In embodiments, the at least one, or at least two, or at least three, or at least four, or at least five a bulky groups are conjugated at or between the positions corresponding to D128 to P134 of SEQ ID NO: 1.

Exemplary suitable bulky groups are independently selected from glycosyl groups, acyl groups, and polymers (e.g., a polyalkylene glycol such as polyethylene glycol (PEG) and poly(2-oxazoline), and poly(2-ethyl-2-oxazoline) (POZ)). Suitable polymers are disclosed in Gouthier and Klok, Polymer-protein conjugates: an enzymatic activity perspective, Polym. Chem., 2010; 1:1352-1373, which is hereby incorporated by reference in its entirety. Suitable polymers are disclosed in U.S. Pat. Nos. 7,595,292; 9,415,115; U.S. Patent Application Publication No. 2010/0239517, which are hereby incorporated by reference in its entirety.

In embodiments, the bulky group is a glycosyl moiety. Conjugation of glycosyl groups to proteins is disclosed in U.S. Pat. No. 7,691,826; U.S. Patent Application Publication No. 2011/0059501, which are each hereby incorporated by reference in its entirety. In embodiments, the bulky group is a polysialic acid moiety. In embodiments, the glycosyl moiety is an N-linked glycosyl moiety. In embodiments, the D1L3 enzyme is mutated to comprise one or more N-linked glycosylation consensus sites between R115 and V146 with respect to SEQ ID NO: 1. In embodiments, the N-linked glycosylation consensus site comprise asparagine (Asn or N)-X-serine (Ser or S)/threonine (Thr or T), wherein X is any amino acid other than proline (Pro or P). In embodiments, the glycosyl moiety is an O-linked glycosyl moiety. In embodiments, the D1L3 enzyme is mutated to comprise one or more O-linked glycosylation consensus sites between R115 and V146 with respect to SEQ ID NO: 1. In embodiments, the O-linked glycosylation consensus site comprises serine (Ser or S) or threonine (Thr or T).

In embodiments, the bulky group is a polyethylene glycol (PEG) moiety. In embodiments, one or more residues of D1L3 is conjugated to a polyethylene glycol (PEG) moiety, resulting in PEGylation of the enzyme. PEGylation of D1L3 can occur at the N-termini, C-termini, an amino acid side chain, the carbon-nitrogen backbone, among other positions of the polypeptide.

In embodiments, one or more amino acids between R115 and V146 corresponding to SEQ ID NO: 1 are PEGylated. In embodiments, one or more amino acids between H120 to S141 (inclusive) corresponding to SEQ ID NO: 1 are PEGylated. In embodiments, one or more amino acids between G125 to V137 (inclusive) corresponding to SEQ ID NO: 1 are PEGylated. In embodiments, one or more amino acids between D128 to P134 (inclusive) corresponding to SEQ ID NO: 1 are PEGylated. In some embodiments, the amino acid corresponding to S131 of SEQ ID NO: 1 is not PEGylated. In embodiments, one or more PEGylated amino acids are selected from lysine, cysteine, histidine, arginine, aspartic acid, glutamic acid, serine, threonine and tyrosine. In embodiments, one or more amino acids suitable for PEGylation are introduced by substitution of one or more amino acids corresponding to R115 and V146 of SEQ ID NO: 1. In embodiments, one or more PEGylated amino acids are lysine (Lys or K) and PEGylation is conducted via amine conjugation. In embodiments, one or more PEGylated amino acids are glutamine (Gln or Q) and PEGylation is conducted via transglutaminase (TGase) mediated enzymatic conjugation. In embodiments, one or more PEGylated amino acids are cysteine (Cys or C) and PEGylation is conducted via thiol conjugation.

In another aspect, the present disclosure provides a D1L3 enzyme comprising a modification that reduces proteolysis after the amino acid corresponding to R95 of SEQ ID NO: 1. In embodiments, the D1L3 enzyme comprises an amino acid sequence that has at least 80%, or at least about 85%, or at least about 90%, or at least about 95%, or at least about 96%, or at least about 97%, or at least about 98%, or at least about 99%, sequence identity to SEQ ID NO: 1.

In embodiments, the D1L3 comprises one or more amino acid substitutions, insertions, and/or deletions within the sequence R92 to E100 of SEQ ID NO: 1. In embodiments, the substitutions, insertions, and/or deletions remove a protease site from D1L3. In embodiments, the one or more modifications disrupt a protease consensus sequence in D1L3. In embodiments, the substitutions, insertions, and/or deletions block access of a protease site to a cleavage site in D1L3. In embodiments, the D1L3 enzyme comprises one, two, three, four, five or more amino acid modifications independently selected from substitutions, deletions, and insertions in the sequence corresponding to R92 to E100 of the SEQ ID NO: 1. In embodiments, the D1L3 enzyme of this disclosure is more stable (resistant to proteolysis) in expression systems, including those described herein (e.g., Pichia pastoris). In embodiments, the D1L3 enzyme is more stable (resistant to proteolysis) in serum.

In embodiments, the amino acid R95 corresponding to SEQ ID NO: 1 is substituted for a different amino acid. In some embodiments, the amino acid corresponding to R95 of SEQ ID NO: 1 is substituted with any amino acid other than a positively charged amino acid such as lysine. In various embodiments, the amino acid corresponding to R95 of SEQ ID NO: 1 is substituted with a polar or an aliphatic amino acid. For example, in some embodiments, the amino acid corresponding to R95 of SEQ ID NO: 1 is substituted with an amino acid selected from serine (Ser or S), threonine (Thr or T), cysteine (Cys or C), glutamine (Glu or Q), asparagine (Asn or N), alanine (Ala or A), Leucine (Leu or L), Isoleucine (Ile or I), or Valine (Val or V).

In embodiments, the modification comprises an amino acid substitution at N96 with respect to SEQ ID NO: 1. In such embodiments, the amino acid corresponding to N96 of SEQ ID NO: 1 is substituted with any amino acid that blocks the protease cleavage at the peptide bond that precedes the amino acid. In some embodiments, the modification comprises a substitution of the amino acid corresponding to N96 of SEQ ID NO: 1 with proline (Pro or P). Other suitable amino acids include Ala, Cys, Gly, Leu, Val, and Ile.

In some embodiments, the modification is the conjugation of a bulky group to the D1L3 enzyme that blocks cleavage of the peptide bond joining amino acids corresponding to R95 and N96 of SEQ ID NO: 1 by a protease. In embodiments, the bulky group is conjugated at the position corresponding to R95 and/or N96 of SEQ ID NO: 1. In embodiments, the bulky group is conjugated at the position corresponding to 1, or 2, or 3, or 4, or 5 amino acids away from R95 and/or N96 of SEQ ID NO: 1. In embodiments, the bulky group is conjugated at the position corresponding to less than about 15, or less than about 12, or less than about 10, or less than about 8, or less than about 6, or less than about 5 amino acids away from R95 and/or N96 of SEQ ID NO: 1. In embodiments, the bulky group is conjugated at or between the positions corresponding to R92 and E100 of SEQ ID NO: 1.

Exemplary suitable bulky groups are as already described, and include glycosyl groups (N- and O-linked) and PEG groups. In embodiments, one or more amino acids corresponding to positions from R95 to E100 of SEQ ID NO: 1 are PEGylated. In embodiments, one or more PEGylated amino acids are selected from lysine, cysteine, histidine, arginine, aspartic acid, glutamic acid, serine, threonine and tyrosine. In embodiments, one or more amino acids suitable for PEGylation are introduced by substitution of one or more amino acids corresponding to R95 and E100 of SEQ ID NO: 1. In embodiments, one or more PEGylated amino acids are lysine (Lys or K) and PEGylation is conducted via amine conjugation. In embodiments, one or more PEGylated amino acids are glutamine (Gln or Q) and PEGylation is conducted via transglutaminase (TGase) mediated enzymatic conjugation. In embodiments, one or more PEGylated amino acids are cysteine (Cys or C) and PEGylation is conducted via thiol conjugation.

In some embodiments, one or more non-cysteine (Cys of C) residues are mutated to a Cys and PEGylated (e.g., by site-specific PEGylation). Such additional sites for PEGylation can be additional proteolytically susceptible sites in wild type D1L3. In some embodiments, these additional sites are surface accessible amino acids, including but not limited to serine amino acids. In some embodiments, the one or more proteolytically susceptible sites are S91, S131, and S253, with respect to SEQ ID NO: 1. See WO 2021/071733, which is hereby incorporated by reference in its entirety. In some embodiments, the D1L3 enzyme comprises one or more substitutions selected from S91C, S131C, and S253C relative to SEQ ID NO: 1, and wherein the amino acid(s) selected from S91C, S131C, and S253C is PEGylated. In these or other embodiments, the amino acid C68 relative to SEQ ID NO: 1, which is believed to be unpaired and accessible in wild-type D1L3, is PEGylated or otherwise modified. For example, the amino acid corresponding to C68 of SEQ ID NO: 1 can be substituted with another amino acid (e.g., Ala, Ser, or Gly, or any amino acid other than Cys), to thereby remove this unpaired Cysteine and/or prevent undesired PEGylation at this position. In still other embodiments, the amino acid C68 relative to SEQ ID NO: 1 forms a disulfide bond with a Cys substituted at a different position. In some embodiments, a Cys is substituted at a position selected from I60, Y87, I89, A103, and L105 relative to SEQ ID NO: 1, and forms a disulfide bond with the side chain of C68. In some embodiments, the modifications to the D1L3 enzyme avoid disulfide scrambling and/or protein misfolding.

In some aspects and embodiments, the disclosure provides a D1L3 enzyme comprising an amino acid sequence that has at least 80% sequence identity to SEQ ID NO: 1 or SEQ ID NO: 2, and wherein the D1L3 enzyme comprises a modification that reduces proteolysis through PEGylation at one or more proteolytically sensitive sites. In such embodiments, the amino acid corresponding to C68 of SEQ ID NO: 1 is PEGylated, substituted with another amino acid, or forms an intramolecular disulfide bond with another amino acid. For example, the one or more proteolytically susceptible sites are Serine amino acids in wild type D1L3, and which are substituted with Cys residues for site-specific PEGylation. In some embodiments, such substitutions are selected from S91C, S131C, and S253C relative to SEQ ID NO: 1. Thus, the D1L3 has an amino acid selected from S91C, S131C, and S253C and which is PEGylated. In various embodiments, the amino acid corresponding to C68 (relative to SEQ ID NO: 1) is also PEGylated. In other embodiments, the amino acid corresponding to C68 of SEQ ID NO: 1 is substituted with another amino acid, so that it is not PEGylated along with the intended PEGylation sites. In still other embodiments, the amino acid corresponding to C68 relative to SEQ ID NO: 1 forms a disulfide bond (intramolecular disulfide bond) with another Cysteine engineered into the D1L3 sequence. In some embodiments, the amino acid corresponding to C68 relative to SEQ ID NO: 1 forms a disulfide bond. with a Cys substituted at a position selected from I60, Y87, I89, A103, and L105 relative to SEQ ID NO: 1.

In embodiments, modifications to D1L3 can be performed, for example and without limitation, as described in U.S. Pat. No. 10,988,746, the contents of which are hereby incorporated in its entirety. For example, mutation of cysteine C68, either by conventional amino acid substitution (e.g., C68A), or by “Building Block” (BB) mutation (e.g., N64_I70delinsHLTAVGK), which can result in a D1L3 variant that maintains chromatin degrading activity. Amino acid sequence alignment shows that cysteine C68 is not conserved among other DNASE1-protein family members, supporting the idea that C68 is not required for enzymatic activity. Furthermore, it was observed that the amino acid substitution of highly conserved cysteine C194 with alanine (C194A), but not the mutation of the highly conserved cysteine C231 with alanine (C231A), resulted in an enzymatically active D1L3 variant. In embodiments, cysteine C68 and C194 can be mutated, PEGylated, or otherwise modified to reduce the risk of extraneous disulfide bond formation during D1L3 production while increasing half-life of D1L3.

In embodiments, unpaired cysteine residues can be introduced into D1L3, for example, non-cysteine residues can be mutated into cysteines, or cysteines can be inserted into motifs (e.g., at a protease site). Such cysteines can also be the site of chemical modification (e.g., PEGylation). In embodiments, one or more naturally-occurring serine residues can be mutated to a cysteine. In embodiments, D1L3 has a C68 modification and a modification (e.g., PEGylation) of one or more other non-naturally occurring cysteine residues, including but not limited to mutations at S91C, S112C, S131C, S253C, S272C, or any combination thereof, among other residues.

In embodiments, the disclosure provides a recombinant D1L3 variant(s) comprising one or more amino acid substitutions or deletions of cysteine residues resulting in reduced intra- and intermolecular cross-linking via disulfide bridges during protein expression. For example, the DNase variant may lack one, two, or three cysteine residues present in the wild-type sequence (e.g., one, two, or three cysteine residues are deleted), or has one or more of such cysteine(s) substituted with other amino acid(s). In embodiments, the one or more cysteine residues are substituted with an amino acid independently selected from Ala, Gly, and Ser, or one or more of the cysteine residues are substituted as part of a BB substitution. In embodiments, the one or more cysteine residues that are substituted is/are not conserved between other members of the D1 protein family (e.g., D1, D1L1, D1L2, and D1L3). In embodiments, the engineered enzyme comprises or further comprises at least one BB substitution from another member of the D1 protein family and/or other point mutation that results in increased protein stability, increased resistance towards degradation by proteases, increased bioavailability, and substantially the same or better DNA and/or chromatin and/or NET-degrading activity (in vitro or in vivo) as compared to the wild-type enzyme. In embodiments, the substitutions and/or modifications include, among other modifications, only a single modification in cysteine residues. In some embodiments, removal of a single cysteine residue is sufficient for significant advantages in manufacturing.

In embodiments, one or more PEGylated amino acids are conjugated with PEG moieties that are independently selected from a linear or branched PEG having molecular weights that are independently selected and in the range of about 2 kDa to about 60 kDa. In embodiments, the PEG moieties have molecular weights that are independently selected from the range of about 5 kDa to about 60 kDa, or about 5 kDa to about 40 kDa, or about 5 kDa to about 30 kDa, or about 10 kDa to about 60 kDa, or about 10 kDa to about 40 kDa, or about 10 kDa to about 30 kDa. In embodiments, the PEGylation or PEG moiety comprises monomethoxypolyethylene glycol (mPEG)-maleimide and/or mPEG-succinimidyl carboxymethyl ester (mPEG-SCM).

In embodiments, cysteines, engineered into the D1L3 sequence or those corresponding to C68 and/or C194 of SEQ ID NO: 1 can be used for site-specific PEGylation. In embodiments, the recombinant D1L3 enzyme comprises one or more polyethylene glycol (PEG) moieties conjugated at the C-terminus. In embodiments, the native amino acid at that position is substituted with an amino acid having a side chain suitable for linkage of the hydrophilic moiety to the enzyme.

In these embodiments, the PEG moiety will provide a half-life extension property and/or improved stability, by providing resistance to proteolysis, reduced disulfide scrambling and/or protein misfolding, and/or a larger hydrodynamic radius to reduce clearance from circulation. In embodiments, the PEG moiety is conjugated through maleimide chemistry, which can be conducted under mild conditions. Other conjugation chemistries are known and may be used, such as vinyl sulfone, dithyopyridine, and iodoacetamide activation chemistries. PEGylation of proteins is further disclosed in WO 2019/036719 and WO 2020/076817, both of which are hereby incorporated by reference in its entirety.

In embodiments, the D1L3 enzyme is a fusion protein with a half-life extending polypeptide. In embodiments, the D1L3 enzyme is fused to a carrier protein, optionally by means of an amino acid linker. The carrier protein is generally a half-life extending polypeptide, such as albumin, transferrin, an Fc domain, XTEN (see U.S. Pat. No. 8,492,530 which is hereby incorporated by reference in its entirety), or elastin-like protein, or a variant thereof. See, e.g., U.S. Pat. No. 9,458,218, which is hereby incorporated by reference in its entirety.

In embodiments, the D1L3 enzyme is fused to an albumin amino acid sequence or domain, i.e., a human albumin or a fragment or variant thereof. See, for example, WO 2015/066550 and U.S. Pat. No. 9,221,896, which are hereby incorporated by reference in their entirety. Albumin can be joined to the D1L3, optionally with an interposed linker, at the N-terminus and/or the C-terminus of the D1L3 enzyme. An exemplary albumin amino acid sequence is provided by SEQ ID NO: 4. In embodiments, the D1L3 enzyme comprises an albumin sequence fused to the N-terminus and/or C-terminus of the mature D1L3 enzyme with an interposed amino acid linker. Linker constructs are described in detail herein.

In embodiments, the albumin amino acid sequence or domain of the fusion protein has at least about 75%, or at least about 80%, or at least about 85%, at least 90%, at least 95%, at least 96%, at least 97%, at least 98%, or at least 99% sequence identity to the reference albumin sequence defined by SEQ ID NO: 4. In embodiments, the albumin amino acid sequence or domain comprises or consists of the reference albumin sequence defined by SEQ ID NO: 4. In embodiments, the albumin amino acid sequence binds to the neonatal Fc receptor (FcRn), e.g., human FcRn. The albumin amino acid sequence may be a variant of wild-type HSA (e.g., as represented by SEQ ID NO: 4). In embodiments, albumin variants have from one to twenty, or from one to ten amino acid modifications independently selected from deletions, substitutions, and insertions with respect to SEQ ID NO: 4. In embodiments, the albumin amino acid sequence is any mammalian albumin amino acid sequence. Various modification to the albumin sequence that enhance its ability to serve as a circulation half-life extending carrier are known, and such modifications can be employed with the present invention. Exemplary modifications to the albumin amino acid sequence are described in U.S. Pat. Nos. 8,748,380, 10,233,228, 10,208,102, and 10,501,524, which are each hereby incorporated by reference in their entireties. Exemplary modifications include one or more (or all) of E505Q, T527M, and K573P as described therein.

In embodiments, the albumin amino acid sequence or domain is a fragment of full-length albumin, as represented by SEQ ID NO: 4. The term “fragment,” when used in the context of albumin, refers to any fragment of full-length albumin or a variant thereof (as described above) that extends the half-life of a D1L3 enzyme to which it is fused or conjugated, relative to the corresponding non-fused D1L3. In embodiments, a fragment of an albumin can refer to an amino acid sequence comprising a fusion of multiple domains of albumin (see, e.g., WO2011/124718), such as domains I and III, and domains II and III. Generally, a fragment of albumin has at least about 100 amino acids, at least about 200, or at least about 300 amino acids of the full-length sequence. In embodiments, the albumin fragment maintains the ability to bind human FcRn.

In embodiments, the D1L3 enzyme is fused to a half-life extending polypeptide (such as an albumin amino acid sequence) through a peptide linker. In embodiments, the linker is a peptide linker, a flexible linker, or a rigid linker. In embodiments, the linker is a physiologically-cleavable linker (e.g., a protease-cleavable linker). In embodiments, the linker is at least about 5 amino acids, at least about 6 amino acids, at least about 7 amino acids, at least about 8 amino acids, at least about 9 amino acids, at least about 10 amino acids, or at least about 15 amino acids. In embodiments, the linker is 5 to 100 amino acids in length, or is 5 to 50 amino acids in length. In embodiments, the linker is from about 10 to about 35 amino acids in length, or from about 15 to about 35 amino acids. In embodiments, the linker is a flexible linker of from 20 to 40 amino acids. Flexible linkers can comprise predominately (or consist of) Gly and Ser amino acid residues.

In embodiments, the D1L3 enzyme is fused to an Fc domain. See, for example, WO 2005047334A1, WO 2004074455A2, US20070269449, all of which are hereby incorporated by reference in their entirety. In embodiments, the human Fc domain is selected from IgG1, IgG2, IgG3, and IgG4. In embodiments, the human Fc domain is a human IgG Fc domain. In embodiments, the Fc domain has at least two heavy chain constant region domains (e.g., CH2 and CH3) and a hinge region. The Fc domain can be joined to the D1L3, optionally with an interposed linker, at the N-terminus and/or the C-terminus of the D1L3 enzyme. In embodiments, the D1L3 enzyme comprises an Fc domain sequence fused to the N-terminus of the mature D1L3 enzyme with an interposed amino acid linker. The peptide linker may be a flexible linker or a rigid linker. In embodiments, the linker is a physiologically-cleavable linker as described herein. In embodiments, the D1L3 enzyme comprises an Fc domain sequence fused to the C-terminus of the D1L3 enzyme, optionally through a linker (e.g., a flexible linker). In embodiments, the D1L3 enzyme (with Fc fused at the C-terminus) has a mutation in one or more amino acids of the BD that reduces or eliminates proteolytic cleavage (without limitation, e.g., substitutions of paired basic amino acids). In embodiments, the D1L3 enzyme (with Fc fused at the C-terminus) has a deletion of the BD to remove the paired basic amino acids in the BD that are otherwise substrates for proteolytic cleavage.

Flexible linkers are predominately or entirely composed of small, non-polar or polar residues such as Gly, Ser and Thr. In some embodiments, the linker contains only Gly and Ser residues. An exemplary flexible linker comprises (Gly_{y}Ser)ₙS_{z} linkers, where y is from 1 to 10 (e.g., from 1 to 5), n is from 1 to about 10, and z is 0 or 1. In embodiments, n is from 3 to about 8, or from 3 to about 6. In exemplary embodiments, y is from 2 to 4, and n is from 3 to 8. Due to their flexibility, these linkers are unstructured. More rigid linkers include polyproline or poly Pro-Ala motifs and α-helical linkers. An exemplary α-helical linker is A(EAAAK)ₙA, where n is as defined above (e.g., from 1 to 10, or 3 to 6). Generally, linkers can be predominately composed of amino acids selected from Gly, Ser, Thr, Ala, and Pro. Exemplary linker sequences contain at least 10 amino acids, and may be in the range of 10 to about 50 amino acids, or about 15 to about 40 amino acids, or about 15 to about 35 amino acids. Exemplary linker designs are provided as SEQ ID NOs: 8 to 15.

In embodiments, the D1L3 enzyme comprises a linker, wherein the amino acid sequence of the linker is predominately glycine and serine residues, or consists essentially of glycine and serine residues. In embodiments, the ratio of Ser and Gly in the linker is, respectively, from about 1:1 to about 1:10, from about 1:2 to about 1:6, or about 1:4. Exemplary linker sequences comprise or consist of S(GGS)₄GSS (SEQ ID NO: 13), S(GGS)₉GSS (SEQ ID NO: 14), (GGS)₉GSS (SEQ ID NO: 15). In embodiments, the linker has at least 10 amino acids, or at least 15 amino acids, or at least 20 amino acids, or at least 25 amino acids, or at least 30 amino acids. For example, the linker may have a length of from 15 to 40 amino acids. In embodiments, longer linkers of at least 15 amino acids can provide improvements in yield upon expression in Pichia pastoris. See PCT/US2019/055178, which is hereby incorporated by reference in its entirety.

In other embodiments, the linker is a physiologically-cleavable linker, such as a protease-cleavable linker. For example, the protease may be a coagulation pathway protease, such as activated Factor XII. In embodiments, the linker comprises the amino acid sequence of Factor XI and/or prekallikrein or a physiologically cleavable fragment thereof. In other embodiments, the linker includes a peptide sequence that is targeted for cleavage by a neutrophil specific protease, such as neutrophil elastase, cathepsin G, and proteinase 3.

In embodiments, the fusion protein is synthesized with an N-terminal signal peptide. The signal peptide may be removed during secretion from the host cell. With respect to expression in Pichia pastoris, the alpha-mating factor (αMF) pre-pro secretion leader from Saccharomyces cerevisiae (SEQ ID NO: 5) may be used for expression. In other embodiments, the signal peptide and propeptide of HSA, which consists of a signal sequence of 24 amino acids (MKWVTFISLLFLFSSAYSRGVFRR; SEQ ID NO: 6) may be used. In embodiments, the human DNASE1L3 Signal Peptide (Q13609) (SEQ ID NO: 7) is used for expression. These elements are cleaved during expression, and are not present in the D1L3 enzyme product.

In any of the embodiments disclosed herein the D1L3 enzyme comprises a deletion of at least 5 amino acids of the C-terminal basic domain, which is also referred herein to as the basic domain (BD), the C-terminal basic domain being defined by SEQ ID NO: 3. In any of the embodiments disclosed herein, the D1L3 enzyme comprises a deletion of the entire C-terminal basic domain. Alternatively, deletions of the BD (e.g., from three to 23 amino acids) can be anywhere in the BD, and not necessarily from the C-terminus of the BD. For example, in embodiments, the D1L3 enzyme has 1, 2, 3, 4, 5, 6, 7, 8, 9, 10, 11, 12, 13, 14, 15, 16, 17, 18, 19, 20, 21, 22, or 23 amino acids deleted from the BD. In embodiments, the D1L3 enzyme has a deletion of at least 1, or at least 3, or at least 5, or at least 8, or at least 12, or at least 15, or at least 18, or at least 21 amino acids from the BD. In embodiments, the D1L3 enzyme has a truncation of at least 1, or at least 3, or at least 5, or at least 8, or at least 12, or at least 15, or at least 18, or at least 21 amino acids from the BD. These deletions can be independently selected from the N-terminal side of the BD, from the C-terminal side of the BD, and internal to the BD. In embodiments, one or more amino acid deletions are within the NLS. In embodiments, the deleted amino acid is the C-terminal serine of the BD. In embodiments, the deletion is sufficient to remove all paired basic amino acids in the BD from the enzyme. See, e.g., WO2021168413, which is hereby incorporated by reference in its entirety.

In embodiments, the BD comprises amino acid substitutions, which may further impact chromatin-degrading activity and/or resistance of the enzyme to proteolysis. For example, the D1L3 enzyme may have from 1 to 20 amino acid substitutions of BD amino acids. In embodiments, the BD contains a substitution of at least three amino acids, or at least five amino acids, or at least 10 amino acids. In embodiments, at least two amino acid substitutions are in the NLS of the BD. In embodiments, one or more paired basic amino acids in the BD are substituted to prevent cleavage. In such embodiments, a more homogeneous enzyme is expressed and secreted, e.g., for recombinant enzyme production.

In embodiments, the D1L3 enzyme comprises an amino acid sequence having at least 80% sequence identity to amino acids 21 to 282 of SEQ ID NO: 1 or amino acids 21 to 252 of SEQ ID NO: 2. In embodiments, the D1L3 enzyme comprises an amino acid sequence having at least 85% sequence identity to amino acids 21 to 282 of SEQ ID NO: 1 or amino acids 21 to 252 of SEQ ID NO: 2. In embodiments, the D1L3 enzyme comprises an amino acid sequence having at least 90% sequence identity to amino acids 21 to 282 of SEQ ID NO: 1 or amino acids 21 to 252 of SEQ ID NO: 2. In embodiments, the amino acid sequence has at least 95%, at least 96%, at least 97%, at least 98%, or at least 99% sequence identity to amino acids 21 to 282 of SEQ ID NO: 1 or amino acids 21 to 252 of SEQ ID NO: 2.

In one aspect, the present disclosure provides an isolated polynucleotide encoding the D1L3 enzyme of any of embodiments disclosed herein. In embodiments, the polynucleotide is DNA. In embodiments, the polynucleotide is an mRNA. In embodiments, the polynucleotide is used for expression of the D1L3 enzyme in host cells suitable for expressing the D1L3 enzyme, and optionally adding glycosyl moieties as described herein for certain embodiments. In other embodiments, the recombinant D1L3 enzyme is expressed and recovered from a recombinant expression system (as described herein), and PEGylated ex vivo using the desired conjugation chemistry.

In one aspect, the present disclosure provides a vector comprising the polynucleotide of any of embodiments disclosed herein. A vector generally comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In embodiments, the vector is a viral vector. Exemplary vectors include autonomously replicating plasmids or a virus (e.g. AAV vectors). The term should also be construed to include non-plasmid and non-viral compounds that facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

In embodiments, host cells are transformed with the DNA molecule or vector. Host cells include cells used for recombinant protein expression, include yeast cell systems, bacterial cell systems, and eukaryotic cell systems including mammalian cells (e.g., CHO cells) and other cell systems conventionally employed. In embodiments that do not require PEGylation, the cell harboring the polynucleotide encoding the D1L3 enzyme can be a cell employed for cell therapy, such as a T cell or stem cell, to thereby produce the D1L3 enzyme in vivo.

In embodiments, the expression vector comprises the nucleic acid encoding the D1L3 enzyme operably linked to an expression control region that is functional in the host cell. The expression control region is capable of driving expression of the operably linked encoding nucleic acid such that the D1L3 enzyme is produced in a desired host cell transformed with the expression vector. Expression control regions are regulatory polynucleotides (sometimes referred to herein as elements), such as promoters and enhancers, that influence expression of an operably linked nucleic acid. An expression control region of an expression vector is capable of expressing operably linked encoding nucleic acid in a desired host cell. In an embodiment, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. In embodiments, the D1L3 enzyme expression is inducible or repressible. Typically, the amount of increase or decrease conferred by such elements is proportional to the amount of signal present; the greater the amount of signal, the greater the increase or decrease in expression.

In embodiments, the viral vector is an adeno-associated viral vector (AAV). In embodiments, suitable AAV-based vectors in the current disclosure have very limited capacity to induce immune responses in humans. The AAV genome is typically built of single-stranded deoxyribonucleic acid (ssDNA), either positive- or negative-sensed, which is about 4.7 kilobases long. The AAV genome comprises inverted terminal repeats (ITRs) at both ends of the DNA strand, and two open reading frames (ORFs): rep and cap. Development of AAVs as gene therapy vectors has eliminated the integrative capacity of the vector by removal of the rep and cap from the DNA of the vector. In embodiments, a gene encoding a D1L3 enzyme disclosed herein, which is operably linked to a promoter, may be inserted between the inverted terminal repeats (ITR). In embodiments, the AAV vector comprising a D1L3 enzyme disclosed herein may form a concatemer in the nucleus after the single-stranded vector DNA is converted by host cell DNA polymerase complexes into double-stranded DNA. In embodiments, the AAV vector comprising a D1L3 enzyme disclosed herein may thus form episomal concatemers in the host cell nucleus. In embodiments, the concatemers may remain intact for the life of the non-dividing host cell. In embodiments, the concatemers may be lost through cell division dividing cells.

Various host cells can be used as recombinant protein expression systems according to the disclosure. In embodiments, the host cell may be a microorganism, a fungal cell, an algal cell, or a plant cell.

In embodiments, the host cell is a microbial cell. The microbial host cell In embodiments may be prokaryotic or eukaryotic. In embodiments, the microbial host cell is a bacteria, and which can be optionally selected from Escherichia spp., Bacillus spp., Corynebacterium spp., Lactococcus spp., and Pseudomonas spp. For example, In embodiments, the bacterial host cell is a species selected from Escherichia coli, Bacillus subtilis, Corynebacterium glutamicum, Pseudomonas fluorescens, and Lactococcus lactis. In embodiments, the bacterial host cell is E. coli. Alternatively, the microbial cell may be a yeast cell, such as but not limited to a species of Saccharomyces, Pichia, Komagataella sp., Kluyveromyces, or Yarrowia, including Saccharomyces cerevisiae, Pichia pastoris, and Yarrowia lipolytica. In embodiments, the host cell is a yeast cell selected from Pichia pastoris, Saccharomyces cerevisiae, Komagataella sp., Kluyveromyces lactis, and Yarrowia lipolytica. In embodiments, the host cell is a fungal cell selected from Aspergillus niger, Trichoderma reesei, and Myceliophthora thermophila. In embodiments, the host cell is Pichia pastoris.

In embodiments, the host cell is a mammalian cell. In embodiments, the host cell is a mammalian cell line. In embodiments, the cell line is selected from NS0 murine myeloma cells, PER.C6 human cells, and Chinese hamster ovary (CHO) cells, baby hamster kidney (BHK21) cells, murine myeloma Sp2/0 cells, human embryonic kidney 293 (HEK293) cells, HT-1080 cells, Hela cells, CAP cells, HKB-11 cells, HuH-7 cells, and a derivative thereof. In embodiments, the cell line is selected from CHO DUXB11, CHO DG44, CHOK1, ExpiCHO and Expi293.

In embodiments, the host cell is an insect cell line. In embodiments, the cell line is Sf9 or a derivative thereof.

In one aspect, the present disclosure provides a pharmaceutical composition comprising the D1L3 enzyme of any of embodiments disclosed herein, and a pharmaceutically acceptable carrier. In embodiments, the pharmaceutical composition is formulated for topical, parenteral, or pulmonary administration. In embodiments, the pharmaceutical composition is formulated for intradermal, intramuscular, intraperitoneal, intraarticular, intravenous, subcutaneous, intraarterial, oral, sublingual, or transdermal administration. In embodiments, the composition is formulated for intravenous or subcutaneous administration.

The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

In still other aspects, the present disclosure provides a process for D1L3 enzyme engineering, by identifying a putative protease cleavage site and modifying D1L3 to block activity of the protease. In embodiments, the modification may be mutation of the putative protease cleavage site. In embodiments, the modification may be the addition of a bulky group at or near the putative protease cleavage site and thereby blocking the access of the protease to the putative protease cleavage site. In embodiments, the bulky group, such as carbohydrates, may be added in host cells when the D1L3 enzyme is biosynthesized. In embodiments, the bulky group, such as polymer conjugates, may be chemically and/or enzymatically added after the D1L3 enzyme is extracted and/or purified.

In other aspects, the present disclosure provides variants of D1L3 enzyme engineered to have advantages in protease resistance, for improving in vivo exposure, e.g., slowing elimination, e.g., extending half-life (e.g., serum half-life), and extending duration of pharmacodynamic activity, as well as reducing proteolysis during recombinant enzyme production. This disclosure identifies, for example, a D1L3 site that is sensitive to proteolysis by a chymotrypsin-like protease that is present in serum and/or produced by mammalian and non-mammalian cell lines. Engineered mutation of these residues can confer these advantages in protease resistance.

In other aspects, the present disclosure provides a method for recombinant production of variants of D1L3 enzyme engineered to have advantages in protease resistance, for improving in vivo exposure, e.g., slowing elimination, e.g., extending half-life (e.g., serum half-life), and extending duration of pharmacodynamic activity, as well as reducing proteolysis during recombinant enzyme production. This disclosure identifies, for example, a D1L3 site that is sensitive to proteolysis by chymotrypsin-like protease that is present in serum and/or produced by mammalian and non-mammalian cell lines. Engineered mutation of D1L3 residues can confer advantages in protease resistance and/or extraneous chemical reaction which can result in breakage of the polypeptide chain (e.g., via oxidation), including resistance against various proteinases, including Arg-C proteinase, Asp-N endopeptidase, Asp-N endopeptidase, chymotrypsin, glutamyl endopeptidase, LysC, LysN, pepsin, proteinase K, thermolysin, trypsin, among other protein degradative enzymes and/or chemicals. In embodiments, the variants of D1L3 disclosed herein have improved resistance to chymotrypsin-like proteinases and proteinase K.

In embodiments, the method for recombinant production of variants of D1L3 enzyme employs a non-mammalian expression system, e.g., a eukaryotic non-mammalian expression system, such as Pichia pastoris. In embodiments, the Pichia pastoris encodes the DNase enzyme with its native signal peptide allowing for secretion from host cells. In embodiments, the expression system is a mammalian cell expression system, such as Chinese Hamster Ovary (CHO) cells. In embodiments, the method for recombinant production of variants of D1L3 enzyme further comprises isolating and/or purifying the D1L3 enzyme and subjecting the isolated and/or purified the D1L3 enzyme to a modification. In embodiments, the modification comprises conjugation of the isolated and/or purified the D1L3 enzyme to a polymer (without limitation, e.g., PEG). In embodiments, the polymer is added to a specific site using the desired conjugation chemistry (without limitation, e.g., maleimide chemistry).

In other aspects, the present disclosure provides a method for treating a subject in need of extracellular chromatin degradation, extracellular trap (ET) degradation and/or neutrophil extracellular trap (NET) degradation. The method comprises administering a therapeutically effective amount of the D1L3 enzyme or composition described herein. Exemplary indications where a subject is in need of extracellular chromatin degradation (including ET or NET degradation) are disclosed in PCT/US18/47084, the disclosure of which is hereby incorporated by reference.

Neutrophils, the predominant leukocytes in acute inflammation, generate neutrophil extracellular traps (NETs), lattices of high-molecular weight chromatin filaments decorated with biologically active proteins and peptides, which immobilize bacteria in wounds. Systemic accumulation of NETs harms tissues and organs due to their cytotoxic, proinflammatory, and prothrombotic activity. Indeed, NETs are frequently associated with inflammatory, ischemic, and autoimmune conditions, including Systemic Lupus Erythematosus (SLE).

In embodiments, the present invention provides a method for treating, preventing, or managing diseases or conditions characterized by the presence or accumulation of NETs. See Jiménez-Alcázar et al., “Host DNases prevent vascular occlusion by neutrophil extracellular traps.” Science 358 (6367): 1202-1206 (2017). A number of stimuli, which sometimes contribute to inflammation and/or pathogenesis, induce NETs. These stimuli include phorbol 12-myristate 13-acetate (PMA), a potent mitogen, lipopolysaccharides (LPS), calcium ionophore A23187, the antibiotic nigericin, which also acts as a potassium ionophore, fungi like Candida albicans, and bacteria like Streptococcus agalactiae (a Group B Streptococcus), Klebsiella pneumoniae and viruses like SARS-CoV2. Leppkes et al. “Vascular occlusion by neutrophil extracellular traps in COVID-19.” EBioMedicine 58 (2020) 102925 (2020); Claushuis et al., “Role of peptidylargininedeiminase 4 in neutrophil extracellular trap formation and host defense during Klebsiella pneumoniae-induced pneumonia-derived sepsis.” J Immunol. 201:1241-1252 (2018); and Kenny et al., “Diverse stimuli engage different neutrophil extracellular trap pathways.” Elife. 6:e24437 (2017). The diseases or conditions characterized by the presence or accumulation of NETs include, but are not limited to, diseases associated with chronic neutrophilia, neutrophil aggregation and/or leukostasis, thrombosis and vascular occlusion, ischemia-reperfusion injury, surgical and traumatic tissue injury, an acute or chronic inflammatory reaction or disease, an autoimmune disease, cardiovascular disease, metabolic disease, systemic inflammation, inflammatory diseases of the respiratory tract, renal inflammatory diseases, inflammatory diseases related to transplanted tissue or hematopoietic stem cell transplantation (e.g. graft-versus-host disease), inflammation caused by viral infections (e.g. COVID-19), and cancer (including leukemia). In embodiments, the present invention provides a method for treating complete or partial vascular or ductal occlusions involving extracellular chromatin, and including NETs.

In embodiments, the method comprising administering the compositions described herein to the subject. In embodiments, the subject is at risk of vascular occlusion involving extracellular chromatin, including chromatin released by cancer cells and injured endothelial cells, among others. Thus, in exemplary embodiments, the subject has cancer (e.g., leukemia or solid tumor). In embodiments, the subject has a hematological cancer selected from multiple myeloma (MM), Hodgkin lymphoma (HL), non-Hodgkin lymphoma (NHL), chronic lymphocytic leukemia (CLL), and acute lymphoblastic leukemia (ALL). In embodiments, the subject has metastatic cancer.

Subjects receiving therapy for cancer (including but not limited to T cell therapies) are at risk of tumor lysis syndrome and/or cytokine release syndrome, which occurs when tumor cells release their contents (including chromatin) into the bloodstream. Tumor lysis syndrome is a complication during the treatment of cancer, where large amounts of tumor cells are killed at the same time by cancer treatment. Tumor lysis syndrome and/or cytokine release syndrome occurs commonly after the treatment of lymphomas and leukemias. In embodiments, the therapy described herein treats, reduces, or prevents tumor lysis syndrome.

In still other embodiments, the subject has an inflammatory disease of the respiratory tract, such as the lower respiratory tract. Exemplary diseases include bacterial and viral infections. In embodiments, the subject has Acute Respiratory Distress Syndrome (ARDS), Acute Lung Injury (ALI), pneumonia, or asthma. Exemplary viral infections in RSV and coronavirus infection (such as SARS, or SARS-CoV-2, e.g., COVID-19 as well as variants thereof).

In still other embodiments, the subject has a disease or condition other than cancer. In embodiments, the disease or condition is an autoimmune or immunological condition, such as those selected from systemic lupus erythematosus (SLE), rheumatoid arthritis, psoriasis, inflammatory bowel disease, celiac sprue, pernicious anemia, scleroderma, Graves' disease, Sjogren syndrome, autoimmune hemolytic anemia (AIHA), myasthenia gravis, cryoglobulinemia, thrombotic thrombocytopeniarpura (TTP), allograft rejection (e.g., transplant rejection of lung, kidney, heart, intestine, liver, pancreas, etc.), pemphigus vulgaris, vitiligo, Hashimoto's disease, Addison's disease, reactive arthritis, and type 1 diabetes.

In embodiments, the subject has SLE. The discovery of NETs raised the speculation that neutrophils may be the predominant source of autoantigens (i.e. dsDNA, chromatin) in SLE (Brinkmann, et al. Neutrophil Extracellular Traps Kill Bacteria. Science, 303(5663): 1532-1545 (2004)). Indeed, autoantibodies such as anti-dsDNA, anti-histone, and anti-nucleosome antibodies bind to NETs, forming pathological ICs. See, for example, Hakkim, et al., Impairment of neutrophil extracellular trap degradation is associated with lupus nephritis, Proceedings of the National Academy of Sciences 107: 9813-9818 (2010). The accumulation of NET-IC breaks immune tolerance via activation of adaptive immune cells that lead to the production of autoantibodies against NET components, forming a vicious cycle of inflammation and autoimmunity. See, for example, Gupta and Kaplan, The role of neutrophils and NETosis in autoimmune and renal diseases. Nat Rev Nephrol. 12(7): 402-13 (2016). Therefore, reducing accumulation of NETs can break the cycle and thus provide an attractive therapeutic strategy for SLE.

In embodiments, the present invention pertains to the treatment of diseases or conditions characterized by deficiency of D1L3, or a deficiency of D1. In some cases, the subject has a mutation (e.g., a loss of function mutation) in a Dnase113 gene or a Dnase1 gene. Such subjects can manifest with an autoimmune disease, such as: systemic lupus erythematosus (SLE), lupus nephritis, scleroderma or systemic sclerosis, rheumatoid arthritis, inflammatory bowel disease, Crohn's disease, ulcerative colitis, and urticarial vasculitis. In some cases, the subject has an acquired inhibitor of D1 (e.g., anti-DNase1-antibody and actin) and/or D1L3 (e.g., anti-Dnase113-antibody). Such subjects can also have an autoimmune or inflammatory disease (e.g., SLE, systemic sclerosis).

In embodiments, the subject has or is at risk of NETs occluding ductal systems. For example, the D1L3 enzymes or compositions disclosed herein can be administered to a subject to treat pancreatitis, cholangitis, conjunctivitis, mastitis, dry eye disease, Stevens-Johnson syndrome, obstructions of vas deferens, or renal diseases.

In embodiments, the subject has or is at risk of NETs accumulating on endothelial surfaces (e.g. surgical adhesions), the skin (e.g. wounds/scarring), or in synovial joints (e.g. gout and arthritis, e.g., rheumatoid arthritis). The D1L3 enzymes and compositions described herein can be administered to a subject to treat a condition characterized by an accumulation of NETs on an endothelial surface such as, but not limited to, a surgical adhesion.

Other diseases and conditions associated with NETs, which the D1L3 enzymes or compositions disclosed herein may be used to treat or prevent, include: ANCA-associated vasculitis, asthma, chronic obstructive pulmonary disease, a neutrophilic dermatosis, dermatomyositis, burns, cellulitis, meningitis, encephalitis, otitis media, pharyngitis, tonsillitis, pneumonia, endocarditis, cystitis, pyelonephritis, appendicitis, cholecystitis, pancreatitis, uveitis, keratitis, disseminated intravascular coagulation, acute kidney injury, acute respiratory distress syndrome, shock liver, hepatorenal syndrome, myocardial infarction, stroke, ischemic bowel, limb ischemia, testicular torsion, preeclampsia, eclampsia, and solid organ transplant (e.g., kidney, heart, liver, and/or lung transplant). Furthermore, the D1L3 enzymes or compositions disclosed herein can be used to prevent a scar or contracture, e.g., by local application to skin, in an individual at risk thereof, e.g., an individual with a surgical incision, laceration, or burn.

In embodiments, the subject has a disease that is or has been treated with wild-type DNases, including D1 and streptodornase. Such diseases or conditions include thrombosis, stroke, sepsis, lung injury, atherosclerosis, viral infection, sickle cell disease, myocardial infarction, ear infection, wound healing, liver injury, endocarditis, liver infection, pancreatitis, primary graft dysfunction, limb ischemia reperfusion, kidney injury, blood clotting, alum-induced inflammation, hepatorenal injury, pleural exudations, hemothorax, intrabiliary blood clots, post pneumatic anemia, ulcers, otolaryngological conditions, oral infections, minor injuries, sinusitis, post-operative rhinoplasties, infertility, bladder catheter, wound cleaning, skin reaction test, pneumococcal meningitis, gout, leg ulcers, cystic fibrosis, Kartagener's syndrome, asthma, lobar atelectasis, chronic bronchitis, bronchiectasis, lupus, primary ciliary dyskinesia, bronchiolitis, empyema, pleural infections, cancer, dry eyes disease, lower respiratory tract infections, chronic hematomas, Alzheimer's disease, and obstructive pulmonary disease.

In embodiments, the subject has a loss of function mutation in one or both D1L3 genes, and may exhibit symptoms of SLE, or may be further diagnosed with clinical SLE.

In embodiments, the composition is administered no more than about weekly, or no more than about every two or three weeks, or no more than about monthly.

Other aspects and embodiments of the invention will be apparent from the following examples.

### Examples

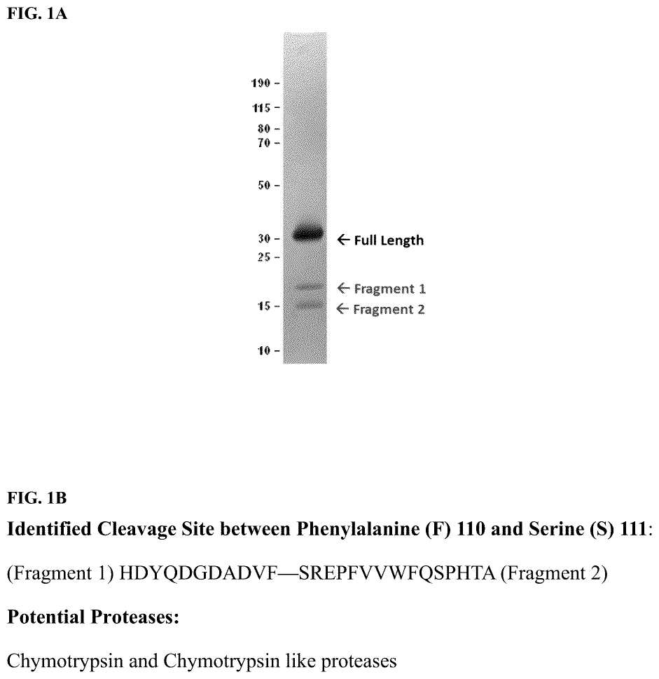

D1L3 enzyme was produced in Pichia pastoris essentially as disclosed in PCT International Application Publication No. WO2019036719, which is hereby incorporated by reference in its entirety. The protein after purification was characterized by SDS-PAGE analysis and Western blot. These experiments show that the protein ran largely as a single band of expected molecular weight (labeled as Full Length in FIG. 1A). However, as also shown in FIG. 1A, two additional bands were observed. Protein from these bands was subjected to amino acid sequencing. Sequence data revealed that the two fragments correspond to two fragments of the D1L3 enzyme. One fragment contained amino acid F130 at the C-terminus, and the second fragment contained amino acid S131 at the N-terminus. Therefore, the two bands represent protein fragments produced by a single cleavage between F130 and S131 of the recombinant protein (FIG. 1B). Based on analysis of structural data, it is believed that this cleavage site is located in a surface-accessible loop. Further, upon analysis of the protease cleavage site, it is believed that the site is susceptible to cleavage by chymotrypsin-like proteases. The sequence DVFSREP (SEQ ID NO: 16) was identified as a putative protease recognition site. Thus, this cleavage site presents a challenge for recombinant protein production as well as for in vivo stability.

Additional mass spec. analysis of a purified D1L3 isoform 1 sample purified after production in Pichia pastoris identified a second protease cleavage site after R95 with respect to SEQ ID NO: 1.

These data suggest, inter alia, that modification of D1L3 may be beneficial for development of D1L3 enzyme therapies. Examples of such modifications include mutation of the protease cleavage site, and/or introduction of bulky groups in this region, such as glycosyl moieties or PEGylation that hinder accessibility of the cleavage site to proteases.

Sequences

SEQ ID NO: 1
Human DNase1L3 (D1L3; Q13609), Amino acid sequence (Signal Peptide; Mature Protein;
Protease cleavage regions):
MSRELAPLLLLLLSIHSALAMRICSFNVRSFGESKQEDKNAMDVIVKVIKRCDIILVMEIKDSN
NRICPILMEKLNRNSRRGITYNYVISSRLGRNTYKEQYAFLYKEKLVSVKRSYHYHDYQDGDAD
VFSREPFVVWFQSPHTAVKDFVIIPLHTTPETSVKEIDELVEVYTDVKHRWKAENFIFMGDFNA
GCSYVPKKAWKNIRLRTDPRFVWLIGDQEDTTVKKSTNCAYDRIVLRGQEIVSSVVPKSNSVFD
FQKAYKLTEEEALDVSDHFPVEFKLQSSRAFTNSKKSVTLRKKTKSKRS
SEQ ID NO: 2
DNASE1-LIKE 3, Isoform 2 (NP_001243489.1): Signal Peptide; Mature Protein; Protease
cleavage region):
MSRELAPLLLLLLSIHSALAMRICSFNVRSFGESKQEDKNAMDVIVKVIKRCDIILVMEIKDSN
NRICPILMEKLNREKLVSVKRSYHYHDYQDGDADVFSREPFVVWFQSPHTAVKDFVIIPLHTTP
ETSVKEIDELVEVYTDVKHRWKAENFIFMGDFNAGCSYVPKKAWKNIRLRTDPRFVWLIGDQED
TTVKKSTNCAYDRIVLRGQEIVSSVVPKSNSVFDFQKAYKLTEEEALDVSDHFPVEFKLQSSRA
FTNSKKSVTLRKKTKSKRS
C-terminal tail of human DNase1L3
SEQ ID NO: 3
Amino acid sequence:
SSRAFTNSKKSVTLRKKTKSKRS
A putative protease recognition site
SEQ ID NO: 16
Amino acid sequence: DVFSREP
SEQ ID NO: 4
Human Serum Albumin (Mature Protein):
DAHKSEVAHRFKDLGEENFKALVLIAFAQYLQQCPFEDHVKLVNEVTEFAKTCVADESAENCDK
SLHTLFGDKLCTVATLRETYGEMADCCAKQEPERNECFLQHKDDNPNLPRLVRPEVDVMCTAFH
DNEETFLKKYLYEIARRHPYFYAPELLFFAKRYKAAFTECCQAADKAACLLPKLDELRDEGKAS
SAKQRLKCASLQKFGERAFKAWAVARLSQRFPKAEFAEVSKLVTDLTKVHTECCHGDLLECADD
RADLAKYICENQDSISSKLKECCEKPLLEKSHCIAEVENDEMPADLPSLAADFVESKDVCKNYA
EAKDVFLGMFLYEYARRHPDYSVVLLLRLAKTYETTLEKCCAAADPHECYAKVFDEFKPLVEEP
QNLIKQNCELFEQLGEYKFQNALLVRYTKKVPQVSTPTLVEVSRNLGKVGSKCCKHPEAKRMPC
AEDYLSVVLNQLCVLHEKTPVSDRVTKCCTESLVNRRPCFSALEVDETYVPKEFNAETFTFHAD
ICTLSEKERQIKKQTALVELVKHKPKATKEQLKAVMDDFAAFVEKCCKADDKETCFAEEGKKLV
AASQAALGL
SIGNAL PEPTIDES
SEQ ID NO: 5
Alpha mating factor (P01149):
MRFPSIFTAVLFAASSALAAPVNTTTEDETAQIPAEAVIGYSDLEGDEDVAVLPFSNSINNGLL
FINTTIASIAAKEEGVS
SEQ ID NO: 6
Human Albumin Secretory Signal Peptide + Propeptide (P02768):
MKWVTFISLLFLESSAYSRGVERR
SEQ ID NO: 7
Human DNASE1L3 Signal Peptide (Q13609):
MSRELAPLLLLLLSIHSALA
LINKER SEQUENCES
SEQ ID NO: 8
GGGGS
SEQ ID NO: 9
GGGGSGGGGSGGGGS
SEQ ID NO: 10
APAPAPAPAPAPAP
SEQ ID NO: 11
AEAAAKEAAAKA
SEQ ID NO: 12
SGGSGSS
SEQ ID NO: 13
SGGSGGSGGSGGSGSS
SEQ ID NO: 14
SGGSGGSGGSGGSGGSGGSGGSGGSGGSGSS
SEQ ID NO: 15
GGSGGSGGSGGSGGSGGSGGSGGSGGSGS

What is claimed is:

1. A DNase1-like 3 (D1L3) enzyme comprising an amino acid sequence that has at least 80% sequence identity to SEQ ID NO: 1 or SEQ ID NO: 2, wherein the D1L3 enzyme comprises a modification that reduces proteolysis after the amino acid corresponding to F130 of SEQ ID NO: 1 or after the amino acid corresponding to R95 of SEQ ID NO: 1.

2. The D1L3 enzyme of claim 1, wherein the D1L3 comprises one or more amino acid substitutions, insertions, and/or deletions within the sequence corresponding to D128 to P134 of SEQ ID NO: 1.

3. The D1L3 enzyme of claim 2, wherein the modification comprises an amino acid substitution at the amino acid corresponding to F130 with a non-aromatic amino acid.

4. The D1L3 enzyme of claim 3, wherein the modification comprises an amino acid substitution at the amino acid corresponding to F130 with a polar, charged, or a non-aliphatic amino acid.

5. The D1L3 enzyme of claim 3, wherein the substitution is selected from serine (Ser or S), threonine (Thr or T), cysteine (Cys or C), glutamine (Glu or Q), and asparagine (Asn or N).

6. The D1L3 enzyme of claim 3, wherein the substitution is selected from lysine (Lys or K), arginine (Arg or R), aspartic acid (Asp or D) and glutamic acid (Glu or E).

7. The D1L3 enzyme of claim 3, wherein the substitution is selected from glycine (Gly or G), alanine (Ala or A), valine (Val or V), isoleucine (Ile or I), proline (Pro or P) and methionine (Met or M).

8. The D1L3 enzyme of any one of claims 1 to 7, wherein the modification comprises an amino acid substitution at the position corresponding to S131 with respect to SEQ ID NO: 1.

9. The D1L3 enzyme of claim 8, wherein the modification comprises a substitution at the position corresponding to S131 of SEQ ID NO: 1 with proline (Pro or P).

10. The D1L3 enzyme of any one of claims 1 to 9, wherein the modification is the conjugation of a bulky group to the D1L3 enzyme that blocks cleavage of the peptide bond joining amino acids corresponding to F130 and S131 of SEQ ID NO: 1 by a protease.

11. The D1L3 enzyme of claim 10, wherein the bulky group is selected from a glycosyl moiety and polyethylene glycol (PEG) moiety.

12. The D1L3 enzyme of claim 11, wherein the bulky group is a glycosyl moiety.

13. The D1L3 enzyme of claim 12, wherein the glycosyl moiety is an N-linked glycosyl moiety.

14. The D1L3 enzyme of claim 12, wherein the glycosyl moiety is an O-linked glycosyl moiety.

15. The D1L3 enzyme of claim 13, wherein the D1L3 enzyme is mutated to comprise one or more N-linked glycosylation consensus sites between R115 and V146 with respect to SEQ ID NO: 1.

16. The D1L3 enzyme of claim 15, wherein the N-linked glycosylation consensus site comprises asparagine (Asn or N)-X-serine (Ser or S)/threonine (Thr or T), wherein X is any amino acid other than proline (Pro or P).

17. The D1L3 enzyme of claim 12, wherein the D1L3 enzyme is mutated to comprise one or more O-linked glycosylation consensus sites between R115 and V146 with respect to SEQ ID NO: 1.

18. The D1L3 enzyme of claim 17, wherein the O-linked glycosylation consensus site comprises serine (Ser or S) or threonine (Thr or T).

19. The D1L3 enzyme of claim 11, wherein the bulky group is a polyethylene glycol (PEG) moiety, optionally wherein one or more amino acids within positions corresponding to R115 to V146 of SEQ ID NO: 1 are PEGylated.

20. The D1L3 enzyme of claim 19, wherein one or more PEGylated amino acids are selected from lysine, cysteine, histidine, arginine, aspartic acid, glutamic acid, serine, threonine and tyrosine, optionally wherein one or more PEGylated amino acids are introduced by substitution of one or more amino acids between R115 and V146 relative to SEQ ID NO: 1.

21. The D1L3 enzyme of claim 20, wherein one or more PEGylated amino acids is lysine (Lys or K) and PEGylation is conducted via amine conjugation.

22. The D1L3 enzyme of claim 20, wherein one or more PEGylated amino acids is glutamine (Gln or Q) and PEGylation is conducted via transglutaminase (TGase) mediated enzymatic conjugation.

23. The D1L3 enzyme of claim 20, wherein one or more PEGylated amino acids is cysteine (Cys or C) and PEGylation is conducted via thiol conjugation.

24. The D1L3 enzyme of claim 23, wherein one or more non-cysteine (Cys of C) residues is mutated to a Cys and PEGylated.

25. The D1L3 enzyme of any one of claims 1 to 24, wherein the D1L3 enzyme comprises a modification that reduces proteolysis after the amino acid corresponding to R95 of SEQ ID NO: 1.

26. The D1L3 enzyme of claim 25, wherein the D1L3 enzyme comprises one or more amino acid substitutions, insertions, and/or deletions within the sequence R92 to E100 of SEQ ID NO: 1.

27. The D1L3 enzyme of claim 26, wherein the D1L3 enzyme comprises one, two, three, four, five or more amino acid modifications independently selected from substitutions, deletions, and insertions in the sequence corresponding to R92 to E100 of the SEQ ID NO: 1.

28. The D1L3 enzyme of claim 27, wherein the amino acid corresponding to R95 of SEQ ID NO: 1 is substituted for a different amino acid.

29. The D1L3 enzyme of claim 28, wherein the amino acid corresponding to R95 of SEQ ID NO: 1 is substituted with any amino acid other than a positively charged amino acid, and optionally a polar or an aliphatic amino acid.

30. The D1L3 enzyme of claim 28, wherein the D1L3 enzyme comprises an amino acid substitution at N96 with respect to SEQ ID NO: 1.

31. The D1L3 enzyme of any one of claims 25 to 30, wherein the D1L3 enzyme comprises conjugation of a bulky group to the D1L3 enzyme that blocks cleavage of the peptide bond joining amino acids corresponding to R95 and N96 of SEQ ID NO: 1 by a protease.

32. The D1L3 enzyme of claim 31, wherein the bulky group is conjugated at the position corresponding to R95 and/or N96 of SEQ ID NO: 1, or the bulky group is conjugated at the position corresponding to 1, or 2, or 3, or 4, or 5 amino acids away from R95 and/or N96 of SEQ ID NO: 1.

33. The D1L3 enzyme of any one of claims 1 to 32, wherein one or more additional proteolytically susceptible sites are modified, optionally by substitution or deletion of one or more serine amino acids.

34. The D1L3 enzyme of claim 33, wherein the one or more additional proteolytically susceptible sites are S91, S131, and S253, with respect to SEQ ID NO: 1.

35. The D1L3 enzyme of claim 34, comprising a substitution selected from S91C, S131C, and S253C relative to SEQ ID NO: 1, and wherein the amino acid selected from S91C, S131C, and S253C is PEGylated.

36. The D1L3 enzyme of any one of claims 1 to 36, wherein the amino acid C68 relative to SEQ ID NO: 1 is PEGylated.

37. The D1L3 enzyme of any one of claims 1 to 36, wherein the amino acid corresponding to C68 of SEQ ID NO: 1 is substituted.

38. The D1L3 enzyme of any one of claims 1 to 37, wherein the amino acid C68 relative to SEQ ID NO: 1 forms a disulfide bond.

39. The D1L3 enzyme of claim 38, wherein the amino acid C68 relative to SEQ ID NO: 1 forms a disulfide bond. with a Cys substituted at a position selected from I60, Y87, I89, A103, and L105 relative to SEQ ID NO: 1.

40. The D1L3 enzyme of any one of claims 1 to 39, wherein one or more PEGylated amino acids are conjugated with PEG moieties that are independently selected from a linear or branched PEG having molecular weights that are independently selected and in the range of about 2 kDa to about 60 kDa.

41. The D1L3 enzyme of claim 40, wherein the PEG moieties have molecular weights that are independently selected from the range of about 5 kDa to about 30 kDa.

42. A DNase1-like 3 (D1L3) enzyme comprising an amino acid sequence that has at least 80% sequence identity to SEQ ID NO: 1 or SEQ ID NO: 2, wherein the D1L3 enzyme comprises a modification that reduces proteolysis through PEGylation at one or more proteolytically sensitive sites, and wherein the amino acid corresponding to C68 of SEQ ID NO: 1 is PEGylated, substituted with another amino acid, or forms an intramolecular disulfide bond with another amino acid.

42. The D1L3 enzyme of claim 42, wherein the one or more proteolytically susceptible sites are Ser amino acids, and which are modified to Cys residues for site-specific PEGylation.

43. The D1L3 enzyme of claim 42, comprising a substitution selected from S91C, S131C, and S253C relative to SEQ ID NO: 1, and wherein the amino acid selected from S91C, S131C, and S253C is PEGylated.

44. The D1L3 enzyme of any one of claims 41 to 43, wherein the amino acid C68 relative to SEQ ID NO: 1 is PEGylated.

45. The D1L3 enzyme of any one of claims 41 to 44, wherein the amino acid corresponding to C68 of SEQ ID NO: 1 is substituted.

46. The D1L3 enzyme of any one of claims 41 to 45, wherein the amino acid C68 relative to SEQ ID NO: 1 forms a disulfide bond.

47. The D1L3 enzyme of claim 46, wherein the amino acid C68 relative to SEQ ID NO: 1 forms a disulfide bond. with a Cys substituted at a position selected from I60, Y87, I89, A103, and L105 relative to SEQ ID NO: 1.

48. The D1L3 enzyme of any one of claims 41 to 47, wherein one or more PEGylated amino acids are conjugated with PEG moieties that are independently selected from a linear or branched PEG having molecular weights that are independently selected and in the range of about 2 kDa to about 60 kDa.

49. The D1L3 enzyme of claim 48, wherein the PEG moieties have molecular weights that are independently selected from the range of about 5 kDa to about 30 kDa.

50. The D1L3 enzyme of any one of the preceding claims, wherein the D1L3 enzyme is a fusion protein with a half-life extending polypeptide.

51. The D1L3 enzyme of claim 50, wherein the half-life extending polypeptide is selected from albumin, transferrin, an Fc, XTEN, and elastin-like protein.

52. D1L3 enzyme of claim 50 or claim 51, further comprising an interposed amino acid linker that joins the D1L3 amino acid sequence with the half-life extending polypeptide.

53. The D1L3 enzyme of claim 52, wherein the amino acid linker is a flexible linker comprising predominately glycine and serine amino acid residues.

54. The D1L3 enzyme of claim 53, wherein the amino acid linker is a rigid linker.

55. The D1L3 enzyme of any one of claims 52 to 54, wherein the amino acid linker comprises a protease cleavage site.

56. The D1L3 enzyme of any one of claims 52 to 55, wherein the linker has at least 15 amino acids.

57. The D1L3 enzyme of any one of claims 52 to 56, wherein the half-life extending polypeptide is albumin.

58. The D1L3 enzyme of claim 57, wherein the albumin is located at the N-terminal side of the D1L3 amino acid sequence.

59. The D1L3 enzyme of claim 57, wherein the albumin is located at the C-terminal side of the D1L3 amino acid sequence.

60. The D1L3 enzyme of any one of claims 57 to 59, wherein the albumin comprises an amino acid sequence that has at least 80%, or at least 90%, or at least 95%, or at least 98% sequence identity to SEQ ID NO: 4.

61. The D1L3 enzyme of claim 60, wherein the albumin comprises an amino acid sequence that has about 100% sequence identity to SEQ ID NO: 4.

62. The D1L3 enzyme of any one of claims 51 to 56, wherein the half-life extending polypeptide comprises an Fc domain.

63. The D1L3 enzyme of any one of the preceding claims, comprising a deletion of at least 5 amino acids of the C-terminal basic domain, the C-terminal basic domain being defined by SEQ ID NO: 3.

64. The D1L3 enzyme of claim 63, comprising a deletion of the entire C-terminal basic domain.

65. An isolated polynucleotide encoding the D1L3 enzyme of any one of the preceding claims.

66. A vector comprising the polynucleotide of claim 65.

66. A host cell comprising the vector of claim 66.

67. The host cell of claim 66, wherein the host cell is a bacterial cell, fungal cell, yeast cell or a mammalian cell.

68. The host cell of claim 67, wherein the host cell is a bacterial cell selected from Escherichia coli, Bacillus subtilis, Corynebacterium glutamicum, Pseudomonas fluorescens, and Lactococcus lactis.

69. The host cell of claim 67, wherein the host cell is a yeast cell selected from Pichia pastoris, Saccharomyces cerevisiae, Komagataella sp., Kluyveromyces lactis, and Yarrowia lipolytica.

70. The host cell of claim 67, wherein the host cell is a fungal cell selected from Aspergillus niger, Trichoderma reesei, and Myceliophthora thermophila.

71. The host cell of claim 67, wherein the host cell is mammalian cell selected from Chinese hamster ovary (CHO), CHO DUXB11, CHO DG44, CHOK1, ExpiCHO, Expi293, NS0 murine myeloma, PER.C6, baby hamster kidney (BHK21), murine myeloma Sp2/0, human embryonic kidney 293 (HEK293), HT-1080, Hela, CAP, HKB-11, and HuH-7, or a derivative thereof.

72. A pharmaceutical composition comprising the D1L3 enzyme of any one of claims 1 to 71, and a pharmaceutically acceptable carrier.

73. The pharmaceutical composition of claim 72, formulated for topical, parenteral, or pulmonary administration.

74. The pharmaceutical composition of claim 72, formulated for intradermal, intramuscular, intraperitoneal, intraarticular, intravenous, subcutaneous, intraarterial, oral, sublingual, or transdermal administration.

75. A method for treating a subject in need of extracellular DNA degradation, extracellular chromatin degradation, extracellular trap (ET) degradation and/or neutrophil extracellular trap (NET) degradation, the method comprising administering a therapeutically effective amount of a composition of any one of claims 72 to 74.

76. The method of claim 75, wherein the subject has a loss of function mutation in a D1L3 gene.

77. The method of claim 75 or 76, wherein the subject has a condition selected from chronic neutrophilia, neutrophil aggregation or leukostasis, thrombosis or vascular occlusion, ischemia-reperfusion injury, surgical or traumatic tissue injury, an acute or chronic inflammatory reaction or disease, an autoimmune disease, cardiovascular disease, metabolic disease, systemic inflammation, inflammatory disease of the respiratory tract, renal inflammatory disease, inflammatory disease related to transplanted tissue and cancer.

78. The method of claim 75 or 76, wherein the subject has, or is at risk of, NETs occluding ductal systems, wherein the condition is optionally selected from pancreatitis, cholangitis, obstructions of vas deferens, and renal disease.

79. The method of claim 75 or 76, wherein the subject has, or is at risk of, NETs accumulating on endothelial surfaces.

80. The method of claim 75 or 76, wherein the subject has an inflammatory disease of the respiratory tract selected from Acute Respiratory Distress Syndrome (ARDS), Acute Lung Injury (ALI), pneumonia, or asthma.

81. The method of claim 75 or 76, wherein the subject has, or is receiving therapy for cancer (including but not limited to T cell therapies).

82. The method of claim 81, wherein the subject is at risk of tumor lysis syndrome and/or cytokine release syndrome.

83. The method of claim 75 or 76, wherein the subject has an inflammatory condition selected from systemic lupus erythematosus (SLE), rheumatoid arthritis, psoriasis, inflammatory bowel disease, celiac sprue, pernicious anemia, scleroderma, Graves' disease, Sjogren syndrome, autoimmune hemolytic anemia (AIHA), myasthenia gravis, cryoglobulinemia, thrombotic thrombocytopenia purpura (TTP), allograft rejection (e.g., transplant rejection of lung, kidney, heart, intestine, liver, pancreas, etc.), pemphigus vulgaris, vitiligo, Hashimoto's disease, Addison's disease, reactive arthritis, and type 1 diabetes.

84. The method of claim 75 or 76, wherein the subject has an autoimmune disease selected from systemic lupus erythematosus (SLE), lupus nephritis, scleroderma or systemic sclerosis, rheumatoid arthritis, inflammatory bowel disease, Crohn's disease, ulcerative colitis, and urticarial vasculitis.

85. The method of claim 84, wherein the subject has SLE.

86. The method of claim any one of claims 75 to 85, wherein the composition is administered no more than about weekly, or no more than about once every two or three weeks, or no more than about monthly.
